(19) United States
(12) Patent Application Publication
(10) Pub. No.: US 2019/0374127 A1
(43) Pub. Date: Dec. 12, 2019

(54) Inversion of Signal Measurements to Generate Medical Images

(71) Applicant: KIMBERLY-CLARK WORLDWIDE, INC., Neenah, WI (US)

(72) Inventor: Joseph R. Feldkamp, Appleton, WI (US)

(21) Appl. No.: 16/483,194

(22) PCT Filed: Feb. 5, 2018
(86) PCT No.: PCT/US2018/016812

§ 371 Date: Aug. 2, 2019

Related U.S. Application Data
(60) Provisional application No. 62/455,756, filed on Feb. 7, 2017.

(51) Int. Cl. A61B 5/05; A61B 5/00

(52) U.S. Cl. CPC A61B 5/0522; G01N 27/023; A61B 5/0033; A61B 5/72

(57) ABSTRACT

Systems and methods for inversion of signal measurements for medical imaging are provided. In one example implementation, a method can include accessing a plurality of signal measurements. The method can include accessing a convolution model defining a relationship between signal measurements and property of the specimen. The method can include determining a property distribution for the specimen by performing an inversion based at least in part on the convolution model and signal measurements. The inversion can based at least in part on a penalized objective function. The penalized objective function having a penalty term. The penalty term for individual solution components can be independently weighted. The method can include generating a property map of the specimen based at least in part on the property distribution; and outputting the property map as an image on a display device.

### RELATED APPLICATION

The present application claims priority to U.S. Patent Application Ser. No. 62/455,756 filed on Feb. 7, 2017, which is incorporated herein in its entirety by reference thereto.

### FIELD

The present disclosure relates generally to the field of medical imaging.

### BACKGROUND

Medical imaging can use various signal measurements to generate an image of tissue, such as human or animal tissue. Magnetic induction tomography imaging can be used to image an electromagnetic property distribution (e.g. conductivity or permittivity) within tissues. More particularly, magnetic induction tomography techniques can provide for the low cost, contactless measurement of electromagnetic properties of tissue based on eddy currents induced in tissues by induction coils placed adjacent to the tissue.

Electromagnetic properties such as conductivity and permittivity vary spatially in tissue due to natural contrasts created by fat, bone, muscle and various organs. As a result, a conductivity or permittivity distribution obtained using magnetic induction tomography imaging techniques can be used to image various regions of the body, including lungs and abdominal regions, brain tissue, and other regions of the body that may or may not be difficult to image using other low cost biomedical imaging techniques, such as ultrasound. In this way, magnetic induction tomography imaging can be useful in the biomedical imaging of, for instance, wounds, ulcers, brain traumas, and other abnormal tissue states.

Existing techniques for magnetic induction tomography imaging typically involve the placement of a large number of coils (e.g. a coil array) near the sample and building an image based upon the measured mutual inductance of coil pairs within the large number of coils placed near the specimen. For instance, an array of source coils and an array of detection coils can be placed adjacent the specimen. One or more of the source coils can be energized using radiofrequency energy and a response can be measured at the detection coils. The conductivity distribution (or permittivity distribution) of the specimen can be determined from the response of the detection coils.

### SUMMARY

Aspects and advantages of embodiments of the present disclosure will be set forth in part in the following description, or may be learned from the description, or may be learned through practice of the embodiments.

One example aspect of the present disclosure is directed to a method of generating an image of a property of a tissue specimen. The method can include accessing, by one or more processors, a plurality of signal measurements. The method can include associating, by the one or more processors, position data with each of the plurality of signal measurements. The method can include accessing, by the one or more processors, a convolution model defining a relationship between signal measurements and property of the specimen. The method can include determining a property distribution for the specimen by performing an inversion based at least in part on the convolution model and signal measurements. The inversion can be based at least in part on a penalized objective function. The penalized objective function can have a penalty term. The penalty term for individual solution components can be independently weighted. The method can include generating a property map of the specimen based at least in part on the property distribution; and outputting the property map as an image on a display device.

These and other features, aspects and advantages of various embodiments will become better understood with reference to the following description and appended claims. The accompanying drawings, which are incorporated in and constitute a part of this specification, illustrate embodiments of the present disclosure and, together with the description, serve to explain the related principles.

### BRIEF DESCRIPTION OF THE DRAWINGS

Detailed discussions of embodiments directed to one of ordinary skill in the art are set forth in the specification, which makes reference to the appended figures, in which:

FIG. 1 depicts an example system for magnetic induction tomography imaging using a MIT device according to example embodiments of the present disclosure;

FIG. 2 depicts a perspective view of an example hand held device according to example embodiments of the present disclosure;

FIG. 3 depicts a side view of an example hand held device according to example embodiments of the present disclosure;

FIG. 4 depicts an example coil for magnetic induction tomography imaging according to example embodiments of the present disclosure;

FIG. 5 depicts example connection traces for a coil for magnetic induction tomography imaging according to example embodiments of the present disclosure;

FIG. 6 depicts a block diagram of an example circuit associated with a coil used for magnetic induction tomography imaging according to example embodiments of the present disclosure;

FIG. 7 depicts a process flow diagram of an example method for magnetic induction tomography imaging according to example embodiments of the present disclosure;

FIG. 8 depicts a plot of a kernel function as a function of distance from the coil along the Z-axis a plurality of different coils according to example embodiments of the present disclosure;

FIGS. 9 and 10 depict virtual phantoms for testing the inversion according to example embodiments of the present disclosure; and

FIGS. 11-15 depict simulation results of images generated based on the virtual phantoms of FIGS. 9 and 10 according to example embodiments of the present disclosure.

### DETAILED DESCRIPTION

Reference now will be made in detail to embodiments, one or more examples of which are illustrated in the drawings. Each example is provided by way of explanation of the embodiments, not limitation of the invention. In fact, it will be apparent to those skilled in the art that various modifications and variations can be made to the embodiments without departing from the scope or spirit of the present disclosure. For instance, features illustrated or described as part of one embodiment can be used with another embodiment to yield a still further embodiment. Thus, it is intended that aspects of the present disclosure cover such modifications and variations.

### Overview

Example aspects of the present disclosure are directed to inversion of signal measurements, by one or more processors, for medical imaging. For instance, a plurality of signal measurements for a specimen can be obtained. A model can be accessed that correlates the measurements with a property distribution across the specimen. An inversion can be performed to determine the property distribution for the specimen based on the model and the signal measurements. An image can be generated based at least in part on the determined property distribution.

According to example aspects of the present disclosure, the inversion can be performed by discretizing the specimen into a finite element mesh. The inversion can solve for a property distribution that best accommodates the coil property measurements according to the model using a penalized objective function. In some embodiments, the penalized objective function can independently weight individual solution components for each node in the finite element mesh to account for biases in the model that correlates the measurements with the property distribution. For instance, a diagonal regularization matrix that uses a kernel function chosen based at least in part on the model can be used to guide weighting of the nodes in the penalty term. The penalized objective function can be put into “standard form” and solved using, for instance, singular value decomposition (SVD) without having to resort to generalized singular value decomposition (GSVD).

Standard form refers to a particular structure of the penalty term that arises when the regularization matrix found in the penalty term is the identity matrix—a diagonal matrix having ones along the entire diagonal of the matrix. An ability to transform a problem into “standard form” greatly improves solution efficiency.

In some embodiments, various strategies can be used to improve the efficiency of the solution. For instance, the penalty term of the objective function can be centered on a uniform solution that best explains loss data in a least squares since. Langrage multipliers can be used to enforce non-negativity and manage a small active set. A global regularization parameter can be assigned from a set of singular values using a discrepancy principle.

Determining a property distribution from signal measurements according to example embodiments of the present disclosure can provide a number of technical effects and benefits. For instance, the inversion can be accomplished more efficiently with less processing time and computing resources, improving the efficiency of the imaging system. For instance, only one SVD computation can be required to complete the inversion. Solving for Lagrange multipliers associated with an active set can be efficient since the system is symmetric and smaller in size than the number of degrees of freedom for an entire problem. In addition, the inversion of signal measurements to generate a property distribution can generate improved images. For instance, the inversion does not over smooth images and can allow for sufficient flexibility to aid improved localization of structure features in the specimen.

Example aspects of the present disclosure will be discussed with reference to inversion of coil property measurements obtained using a single coil in a magnetic induction tomography imaging system for purposes of illustration and discussion. Those of ordinary skill in the art, using the disclosures provided herein, will understand that the aspects of the present disclosure can be used for the inversion of signal measurements in other imaging technology, such as CT X-ray imaging and other tomographic imaging technology, such as diffuse optical tomography.

Magnetic induction tomography imaging of specimens, such as human tissue specimens, can be obtained using a single coil. A plurality of coil property measurements can be obtained using the single coil at a plurality of different discrete locations relative to the specimen. The single coil can be designed to be relatively easy to be placed in many different positions/orientations relative to the specimen. A three-dimensional electromagnetic property map, such as a three-dimensional conductivity map or a three-dimensional permittivity map, can be generated from the plurality of coil property measurements obtained using the single coil. In this way, a simple and cost effective way of imaging tissue can be provided using contactless coil property measurements by a single coil.

More particularly, a coil-loss model can be used to define a relationship between coil loss measurements obtained using a single coil and an electromagnetic property distribution of a specimen. In one implementation, the coil-loss model is a quantitative analytical coil-loss model that describes the real part of a change in impedance (e.g. ohmic loss) of a single planar multi-loop coil, having a plurality of concentric conductive loops, resulting from induced eddy currents when excited with RF energy and placed near to arbitrarily shaped objects with arbitrary three-dimensional conductivity distributions. The coil-loss model can include a convolution integral that convolves an electrical conductivity distribution with a sampling function (e.g., a kernel).

Using the coil-loss model, a three-dimensional electromagnetic property map can be generated for human tissue using the plurality of coil property measurements obtained using the single coil. For instance, a plurality of coil loss measurements obtained for the specimen can be accessed. Each coil property measurement can be associated with one of a plurality of discrete locations relative to the specimen. Position data can be associated with each coil property measurement. The position data can be indicative of the position and orientation of the single coil when the measurement was performed.

Once a plurality of coil property measurements and associated position data have been obtained, inversion of the obtained coil property measurements can be performed using the coil-loss model to obtain a three-dimensional electromagnetic property map indicative of the electromagnetic property distribution (e.g. conductivity distribution) of the specimen leading to the plurality of obtained measurements.

In one particular implementation, the inversion can be performed by discretizing the specimen into a finite element mesh. The inversion can solve for a conductivity distribution for points or nodes in the mesh that best accommodates the coil-loss model and the conductivity measurements. The solved conductivity distribution can be output as the three-dimensional conductivity map for the specimen. The three-dimensional conductivity map can be provided as an image for display on a display device

According to example aspects of the present disclosure, the inversion can be accomplished in a way that is particularly well suited to the convolution integral of the coil-loss model. For instance, an invertible diagonal regularization matrix for the penalty term of the penalized objective function can be chosen that uses a kernel function associated with the convolution integral to guide how individual solution components are weighted in the penalty term of the penalized objective function. In some embodiments, the penalty term can quadratic about a uniform conductivity solution that is simply the single conductivity value that best predicts the measured coil loss. Because the regularization matrix is invertible, the penalized objective function can be put into “standard form,” paving the way to use SVD rather than GSVD.

In some embodiments, non-negativity can be enforced by a combination of Lagrange multipliers and an active set so that the number of iterations tends to be small. Because the measurement error is known from experiment, a discrepancy principle can be used to help guide the selection of a global regularization parameter. More particularly, the regularization parameter can be stepped through a partial sequence of singular values, progressing from largest to smallest, until a solution error is close to measurement error.

In this way, only a single SVD computation can be required to complete the inversion. Solving for the Lagrange multipliers associated with the active set is efficient since the system is symmetric and much smaller in size than the number of degrees of freedom for the entire problem. As a result, the inversion is efficient, does not over smooth images, and allows flexibility to aid improved location of structural features.

One example embodiment of the present disclosure is directed to a method for imaging a tissue specimen using magnetic induction tomography. The method includes accessing, by one or more processors, a plurality of coil property measurements obtained for a specimen using a single coil. Each of the plurality of coil property measurements can be obtained with the single coil at one of a plurality of discrete locations relative to a specimen. The method can include associating, by the one or more processors, coil position data with each of the plurality of coil property measurements. The coil position data can be indicative of the position and orientation of the single coil relative to the specimen for each coil property measurement. The method includes accessing, by the one or more processors a coil-loss model defining a relationship between coil property measurements obtained by the single coil and an electromagnetic property of the specimen. The method includes determining, by the one or more processors, an electromagnetic property distribution for the specimen by performing an inversion based at least in part on the coil-loss model and the coil property measurement. The inversion is based at least in part on a penalized objective function. The penalized objective function ahs a penalty term. The penalty terms for individual nodes in a finite element mesh are independently weighted. The method can include generating, by the one or more processors, an electromagnetic property map of the specimen using the coil-loss model based at least in part on the electromagnetic property distribution.

Variations and modifications can be made to this example embodiment. For instance, in some embodiments, the penalty terms for individual nodes are independently weighted based on a distance along a perpendicular axis relative to the coil from a target boundary. In some embodiments, the penalty terms are weighted to account for bias in the coil-loss model.

In some embodiments, the inversion uses a regularization matrix to weigh the penalty term for individual nodes based on a kernel function associated with the coil-loss model. The regularization matrix can be a diagonal regularization matrix. The penalty term can be quadratic about a uniform electromagnetic property solution. In some embodiments, the inversion includes converting the penalized objective function to stand form to generate a standard form objective function using an inverse of the regularization matrix.

In some embodiments, the inversion applies singular value decomposition (SVD) to the standard form objective function to generate an SVD objective function. The inversion can expand the SVD objective function using Lagrange multipliers to achieve a Lagrange objective function. The inversion can enforce a non-negative constraint using one or more Lagrange multipliers. The inversion can step a global regularization parameter associated with the penalty term through a sequence of singular values until a solution error is within a threshold of a measurement error. The sequence of singular values can progress from largest to smallest.

In some embodiments, each of the plurality of coil property measurements includes a coil loss measurement indicative of a change in impedance of the single coil resulting from eddy currents induced in the specimen when the single coil is placed adjacent to the specimen and energized with radio frequency energy. In some embodiments, the electromagnetic property is conductivity. In some embodiments the method can include outputting the electromagnetic property map as an image on a display device.

Another example aspect of the present disclosure is directed to a method for generating an image of a property of a tissue specimen. The method can include accessing, by one or more processors, a plurality of signal measurements. The method can include associating position data with each of the plurality of signal measurements. The method can include accessing a convolution model defining a relationship between signal measurements and a property of the specimen. The method can include determining a property distribution for the specimen by performing an inversion based at least in part on the convolution model and signal measurements. The inversion can be based at least in part on a penalized objective function. The penalized objective function can have a penalty term. The penalty term for individual solution components can be individually weighted. The method can include generating a property map of the specimen based at least in part on the property distribution. The method can include outputting the property map as an image on a display device.

Variations and modifications can be made to this example embodiment. For instance, in some embodiments, the penalty terms for individual nodes can be independently weighted based on a distance along a perpendicular axis relative to a measurement apparatus for a target boundary. In some embodiments, the penalty terms for individual nodes are independently weighted to account for bias in the convolution model.

In some embodiments, the inversion uses a regularization matrix to weight the penalty term for individual nodes based on a kernel function associated with the convolution mode. The regularization matrix can be a diagonal regularization matrix. The penalty term can be quadratic about a uniform property solution. The inversion can include converting the penalized objective function to standard form to generate a standard form objective function using an inverse of the regularization matrix.

In some embodiments, the inversion applies singular value decomposition (SVD) to the standard form objective function to generate an SVD objective function. The inversion can expand the SVD objection function using Lagrange multipliers to achieve a Lagrange objective function. The inversion can enforce a non-negative constraint using one or more Lagrange multipliers. The inversion can determine a solution for the Lagrange objective function using an active set and a plurality of iterations. The inversion can step a global regularization parameter associated with the penalty term through a sequence of singular values until a solution error is within a threshold of a measurement error. The sequence of singular values progressing from largest to smallest.

Other example embodiments of the present disclosure are directed to system. The systems can include one or more processors and one or more memory devices. The one or more processors can store computer-readable instructions that when executed by the one or more processors cause the one or more processors to perform operations, such as one or more operations of any of the methods described herein.

For instance, in one example embodiment, a system can include

### Example Systems for Magnetic Induction Tomography Imaging

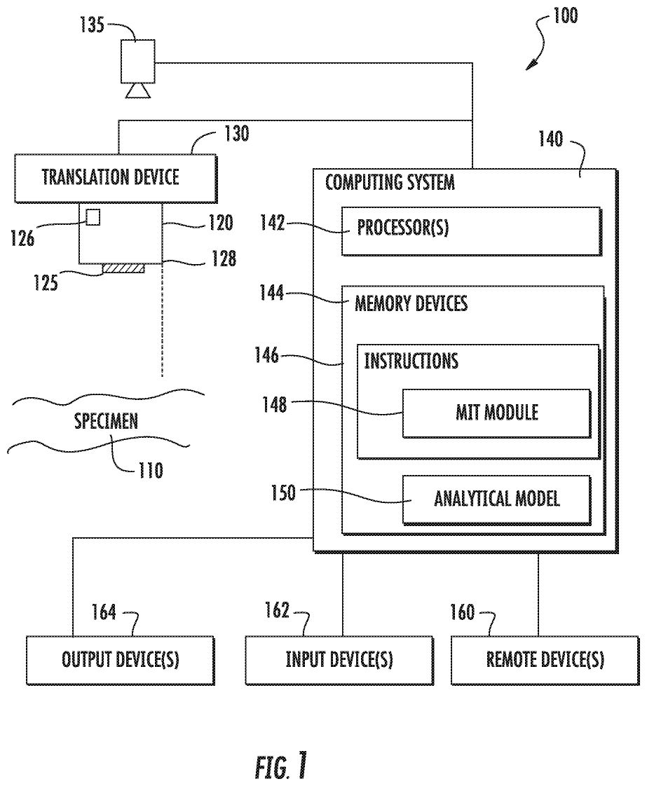

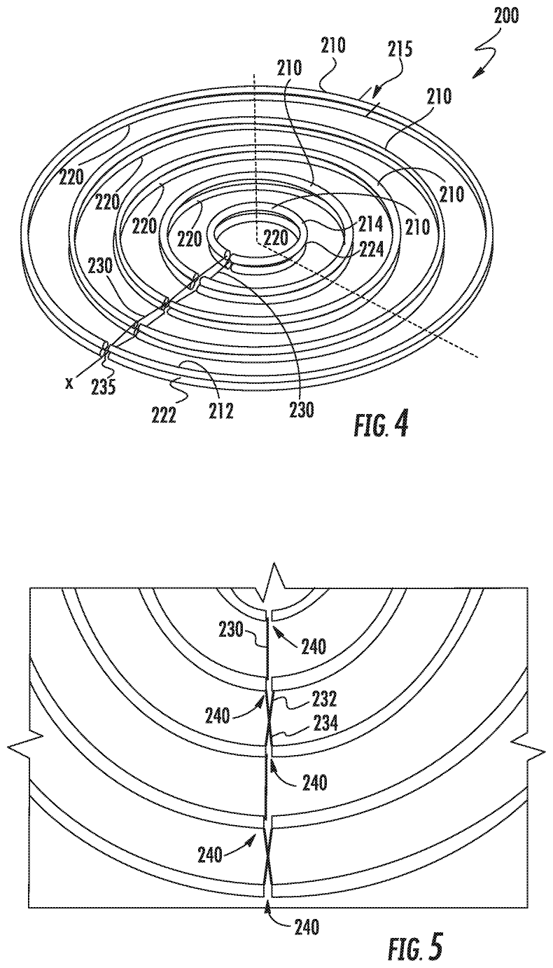

FIG. 1 depicts an example system 100 for magnetic induction tomography imaging of a specimen 110, such as a human tissue specimen. The system 100 includes a coil device 120 having a single coil 125 for obtaining coil property measurements for magnetic induction tomography imaging according to example aspects of the present disclosure. The coil 125 can be a single coil having a plurality of concentric conductive loops disposed in one or more planes on a printed circuit board. One example coil design for magnetic induction tomography imaging according to example aspects of the present disclosure will be discussed in more detail below with reference to FIGS. 4 and 5 below.

The coil device 120 of FIG. 1 can include an RF energy source (e.g. an oscillator circuit) configured to energize the coil 125 with RF energy at a set frequency (e.g. 12.5 MHz) when the coil 125 is placed adjacent to the specimen 110. The energized coil 125 can generate magnetic fields, which can induce eddy currents in the specimen 110. These induced eddy currents in the specimen can cause a coil loss (e.g. a change in impedance) of the coil 125. The coil device 120 can include circuitry (e.g. a measurement circuit) for determining the coil loss associated with the coil 125 during a coil property measurement at a particular location relative to the specimen 110.

Coil property measurements can be obtained using the single coil 125 of the coil device 120 while the coil device 120 is positioned at a variety of different locations and orientations relative to the specimen 110. The collected coil property measurements can be provided to the computing system 140 where the coil property measurements can be analyzed to generate a three-dimensional electromagnetic property map of the specimen 110, such as a three-dimensional conductivity map or a three-dimensional permittivity map of the specimen 110.

In some particular implementations, the coil device 120 can be mounted to a translation device 130. The translation device 130 can be a robotic device controlled, for instance, by the computing system 140 or other suitable control device, to translate the coil device 120 along x-, y-, and -z axes relative to the specimen 110 in order to position the coil 125 at a plurality of different discrete locations relative to the specimen 110. The coil device 120 can be controlled (e.g. by the computing system 140) to obtain a coil property measurement using the coil 125 at each of the plurality of discrete locations.

Alternatively, the coil device 120 can be manually positioned at the plurality of discrete locations for performance of the coil property measurement. For instance, a medical professional can manually position a hand held coil device 120 relative to the specimen 110 to obtain coil property measurements at a plurality of discrete locations relative to the specimen 110.

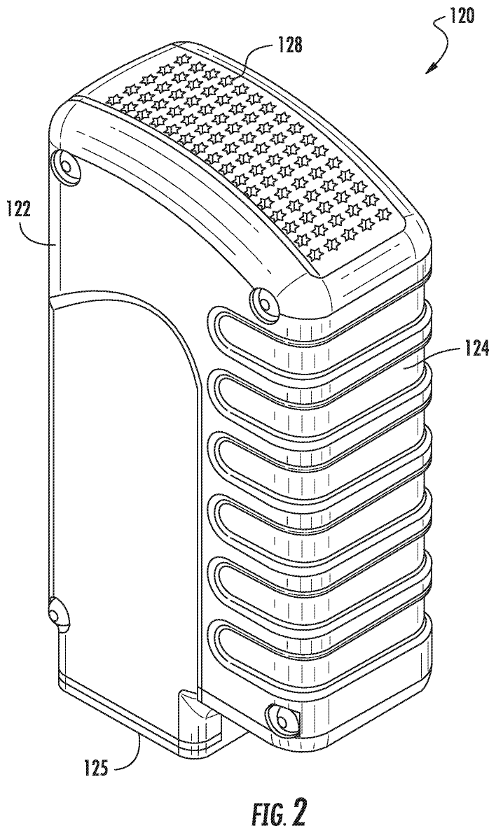

FIG. 2 depicts a perspective view on one example embodiment of a hand held device 120 for magnetic induction tomography imaging according to example embodiments of the present disclosure. As shown, the hand held device 120 includes a housing 122 for storing and protecting various components (e.g., electrical components) of the hand held device 120 used to support acquisition of coil measurements using sensing unit 125.

The example hand held device 120 of FIG. 2 includes a form factor to facilitate holding the hand held device 120 by hand during acquisition of coil measurements. For instance, the hand held device 120 includes a grip portion 124. As illustrated in FIG. 2, the grip portion 124 can include one or more grooves or channels to facilitate grasping or holding the hand held device by hand 120. The hand held device 120 further includes a form factor such that the location of a hand grasping the housing when in operation is separated a threshold distance from the single coil of the sensing unit 125. For instance, the grip portion 124 can be located in the range of about 0.5 inches to about 6 inches away from the sensing unit 125, such as about 2 inches to 4 inches away from the sensing unit, such as about 3 inches away from the sensing unit. In this way, interference between a technician's hand and the sensing unit 125 can be reduced while performing measurements with the hand held device 120.

The hand held device 120 depicts one example form factor according to example embodiments of the present disclosure to facilitate holding the device by hand. Those of ordinary skill in the art, using the disclosures provided herein, should understand that other form factors are contemplated. For instance, the hand held device 120 can have a housing having a first portion that has a first shape adapted to conform to the sensing unit 125 and a second portion that is a different shape (e.g., a cylindrical shape) that is adapted to be held by hand during operation.

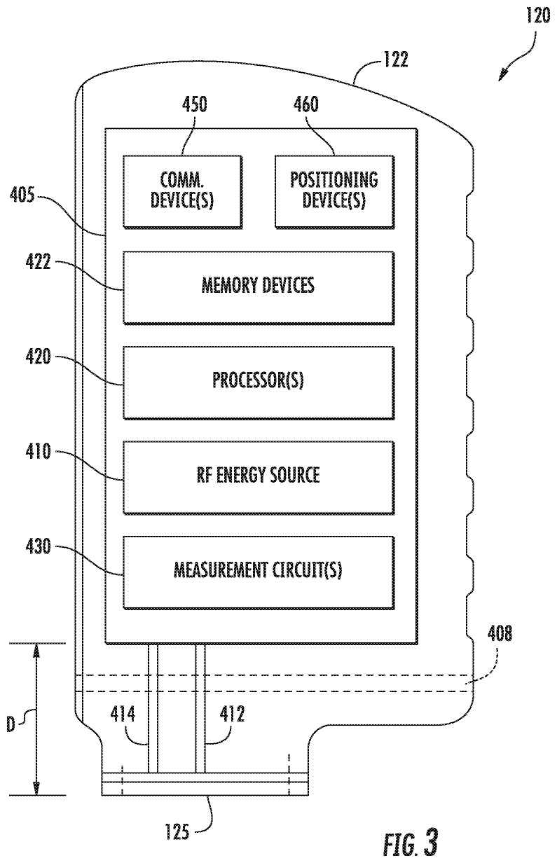

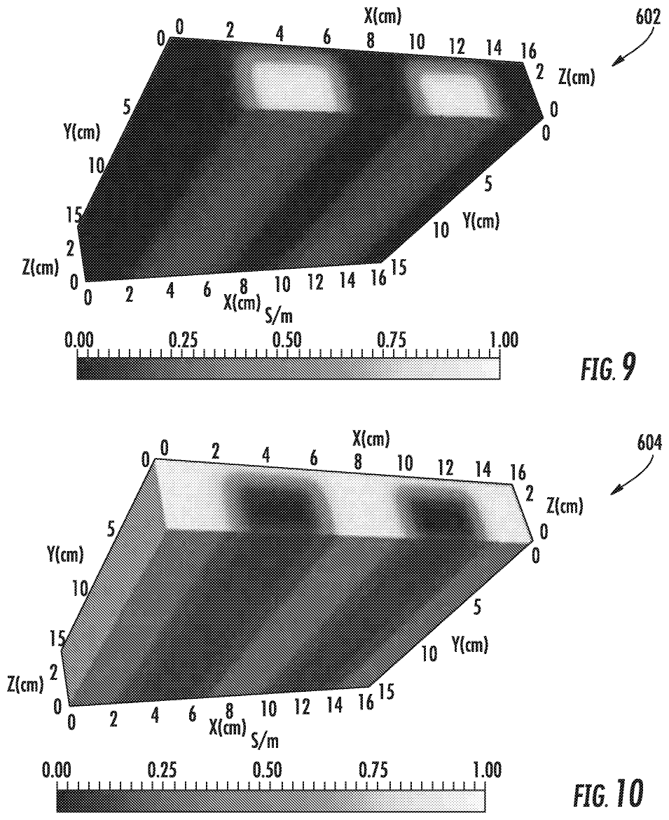

As shown in FIG. 3, the hand held device 120 can include one or more electrical components to support operation of the hand held device 120. The one or more electrical components can include a power source such as a battery (not shown), an RF energy source 410, processor(s) 420, memory device(s) 422, measurement circuit(s) 430, communication device(s) 450, and positioning device(s) 460. Operation of selected of the above electrical components will be discussed in more detail with reference to FIG. 9 below.

Referring to FIG. 3, the RF energy source 410 (e.g., an oscillator circuit) can be configured to generate RF energy for energizing the coil of the sensing unit 125. The processor(s) 420 can be configured to control various aspects of the circuit 400 as well as to process information obtained by the circuit 400 (e.g., information obtained by measurement circuit 430). The processor(s) 420 can include any suitable processing device, such as digital signal processor, microprocessor, microcontroller, integrated circuit or other suitable processing device. The memory devices 422 can be configured to store information and data collected by the hand held device 120. For instance, the memory devices 422 can be configured to store coil measurements obtained by the sensing unit 125. The memory devices 422 can include single or multiple portions of one or more varieties of tangible, non-transitory computer-readable media, including, but not limited to, RAM, ROM, hard drives, flash drives, optical media, magnetic media or other memory devices. The measurement circuit 430 can be configured to obtain coil measurements of the single coil of the sensing unit 125. Details of an example measurement circuit are discussed with reference to FIG. 9 below.

The positioning device(s) 460 of FIG. 3 can include circuitry for supporting one or more sensors used to determine the position and/or orientation of the hand held device 120 when performing coil measurements. For instance, the positioning device(s) 460 can include motion sensors (e.g., accelerometers, compass, magnetometers, gyroscopes, etc.) and other suitable sensors that provide signals indicative of the orientation of the hand held device 120. Further, the hand held device 120 can include depth sensors (e.g., laser sensors, infrared sensors, image capture devices) that can be used to determine a depth or distance of the hand held device 120 to a specimen. Signals from the positioning device(s) 460 can be used in determining a position and/or orientation associated with each coil measurement.

The communication device(s) 450 can be used to communicate information from the hand held device 120 to a remote location, such as a remote computing device. The communication device(s) can include, for instance, transmitters, receivers, ports, controllers, antennas, or other suitable components for communicating information from the hand held device 120 over a wired and/or wireless network.

The various electrical components supporting operation of the hand held device 120 can be disposed on a printed circuit board 405 within the housing 122 of the hand held device 120. As illustrated, in FIG. 3, the one or more electrical components can be separated a threshold distance D from the sensing unit 125 so as to reduce interference between the one or more electrical components and the sensing unit 125. In particular embodiments, the threshold distance D can be in the range of about 0.5 inches to about 4 inches, such as about 2 inches to 3 inches away, such as about 2 inches.

As shown in FIG. 3, the hand held device 120 can further include a shield 408. The shield 408 can be manufactured from a conductive material or high dielectric constant, non-lossy material. The shield 408 can separate the sensing unit 125 from the electrical components supporting operation of the hand held device 120 to further reduce electromagnetic interference between the electrical components and the sensing unit 125. Conductive paths 412 and 414 passing through the shield 408 can be used to communicate signals from the sensing unit 125 to the electrical components supporting operation of the hand held device 120. One or more of the electrical components supporting operation of the hand held device and other components of the magnetic induction tomography system can be located at a location remote from the hand held device 120.

Referring to FIG. 1, to generate an accurate three-dimensional electromagnetic property map of the specimen 110, position data needs to be associated with each of the obtained coil property measurements. The position data can be indicative of the position (e.g. as defined by an x-axis, y-axis, and a z-axis relative to the specimen 110) of the coil 125 as well as an orientation of the coil 125 (e.g. a tilt angle relative to the specimen 110). When using a translation device 130 to position the coil 125, the position and orientation of the coil 125 can be determined based at least in part on positioning control commands that control the translation device 130 to be positioned at the plurality of discrete locations.

In one embodiment of the present disclosure, images captured by a camera 135 positioned above the specimen 110 and the coil device 120 can be processed in conjunction with signals from various sensors associated with the coil device 120 to determine the position data for each coil property measurement. More particularly, the coil device 120 can include one or more motion sensors 126 (e.g. a three-axis accelerometer, gyroscope, and/or other motion sensors) and a depth sensor 128 The orientation of the single coil 125 relative to the surface can be determined using the signals from the motion sensors 126. For instance, signals from a three-axis accelerometer can be used to determine the orientation of the single coil 125 during a coil property measurement.

The depth sensor 128 can be used to determine the distance from the single coil to the specimen 110 (e.g. the position along the z-axis). The depth sensor 128 can include one or more devices configured to determine the location of the coil 125 relative to a surface. For instance, the depth sensor 128 can include one or more laser sensor devices and/or acoustic location sensors. In another implementation, the depth sensor 128 can include one or more cameras configured to capture images of the specimen 110. The images can be processed to determine depth to the specimen 110 using, for instance, structure-from-motion techniques.

Images captured by the camera 135 can be used to determine the position of the coil 125 along the x-axis and y-axis. More particularly, the coil device 120 can also include a graphic located on a surface of the coil device 120. As the plurality of coil property measurements are performed, the image capture device 135 can capture images of the graphic. The images can be provided to the computing system 140, which can process the images based on the location of the graphic to determine the position along the x-axis and y-axis relative to the specimen 110. In particular implementations, the camera 135 can include a telecentric lens to reduce error resulting from parallax effects.

In some embodiments, magnetic induction tomography device can include one or more reflective components mechanically coupled to the device. The reflective components can be, for instance, a plurality of reflective spheres or other reflective bodies (e.g., cubes, cylinders, trapezoids, etc.). An optical tracking sensor located proximate to an area where the hand held magnetic induction tomography device is in use can generate signals indicative of the location of one or more reflective components based on a reflection of optical signals (e.g., infrared signals or other optical signals) from the plurality of reflective spheres.

The computing system 140 can receive the coil property measurements, together with coil location and orientation data, and can process the data to generate a three-dimensional electromagnetic property map of the specimen 110. The computing system 140 can include one or more computing devices, such as one or more of a desktop, laptop, server, mobile device, display with one or more processors, or other suitable computing device having one or more processors and one or more memory devices. The computing system 140 can be implemented using one or more networked computers (e.g., in a cluster or other distributed computing system). For instance, the computing system 140 can be in communication with one or more remote devices 160 (e.g. over a wired or wireless connection or network).

The computing system 140 includes one or more processors 142 and one or more memory devices 144. The one or more processors 142 can include any suitable processing device, such as a microprocessor, microcontroller, integrated circuit or other suitable processing device. The memory devices 144 can include single or multiple portions of one or more varieties of tangible, non-transitory computer-readable media, including, but not limited to, RAM, ROM, hard drives, flash drives, optical media, magnetic media or other memory devices. The computing system 140 can further include one or more input devices 162 (e.g. keyboard, mouse, touchscreen, touchpad, microphone, etc.) and one or more output devices 164 (e.g. display, speakers, etc.).

The memory devices 144 can store instructions 146 that when executed by the one or more processors 142 cause the one or more processors 142 to perform operations. The computing device 140 can be adapted to function as a special-purpose machine providing desired functionality by accessing the instructions 146. The instructions 146 can be implemented in hardware or in software. When software is used, any suitable programming, scripting, or other type of language or combinations of languages may be used to implement the teachings contained herein.

As illustrated, the memory devices 144 can store instructions 146 that when executed by the one or more processors 142 cause the one or more processors 142 to implement a magnetic induction tomography (“MIT”) module 148. The MIT module 148 can be configured to implement one or more of the methods disclosed herein for magnetic induction tomography imaging using a single coil, including the inversion of coil property measurements to generate a property map.

The one or more memory devices 144 of FIG. 1 can also store data, such as coil property measurements, position data, three-dimensional electromagnetic property maps, and other data. As shown, the one or more memory devices 144 can store data associated with an analytical coil-loss model 150. The analytical coil-loss model 150 can define a relationship between coil property measurements obtained by a single coil and an electromagnetic property distribution of the specimen 110. Features of an example analytical coil-loss model will be discussed in more detail below.

MIT module 148 may be configured to receive input data from input device 162, from coil device 120, from translation device 130, from data that is stored in the one or more memory devices 144, or other sources. The MIT module 148 can then analyze such data in accordance with the disclosed methods, and provide useable output such as three-dimensional electromagnetic property maps to a user via output device 164. Analysis may alternatively be implemented by one or more remote device(s) 160.

The technology discussed herein makes reference to computing systems, servers, databases, software applications, and other computer-based systems, as well as actions taken and information sent to and from such systems. One of ordinary skill in the art, using the disclosures provided herein, will recognize that the inherent flexibility of computer-based systems allows for a great variety of possible configurations, combinations, and divisions of tasks and functionality between and among components. For instance, processes discussed herein may be implemented using a single computing device or multiple computing devices working in combination. Databases and applications may be implemented on a single system or distributed across multiple systems. Distributed components may operate sequentially or in parallel.

### Example Quantitative Analytical Coil-Loss Model for a Single Coil

An example quantitative analytical coil-loss model for obtaining a three-dimensional conductivity map from a plurality of coil property measurements obtained by a MIT device will now be set forth. The quantitative coil-loss model is developed for an arbitrary conductivity distribution, but with permittivity and magnetic permeability treated as spatially uniform. The quantitative analytical coil-loss model was developed for a coil geometry that includes a plurality of concentric circular loops, all lying within a common plane and connected in series, with the transient current considered to have the same value at all points along the loops. A conductivity distribution is permitted to vary arbitrarily in space while a solution for the electric field is pursued with a limit of small conductivity (<10 S/m). Charge free conditions are assumed to hold, whereby the electrical field is considered to have zero divergence. Under these conditions, fields are due only to external and eddy currents.

The quantitative analytical coil-loss model can correlate a change in the real part of impedance (e.g., ohmic loss) of the coil with various parameters, including the conductivity distribution of the specimen, the position and orientation of the single coil relative to the specimen, coil geometry (e.g. the radius of each of the plurality of concentric conductive loops) and other parameters. One example coil-loss model is provided below:

$$- δ {Z}_{re} = \frac{{μ}^{2} {ω}^{2}}{4 {π}^{2}} {\sum }_{j , k} \sqrt{{ρ}_{j} {ρ}_{k}} \int {d}^{3} x \frac{\breve{σ} ( \vec{r} )}{ρ} {Q}_{\frac{1}{2}} ( {η}_{j} ) {Q}_{\frac{1}{2}} ( {η}_{k} )$$

−δZᵣₑ is the coil property measurement (e.g., the real part of the impedance loss of the coil). μ is the magnetic permeability in free space. ω is the excitation frequency of the coil. ρₖ and ρⱼ are the radii of each conductive loop j and k for each interacting loop pair j,k. The function Q_{1/2} is known as a ring function or toroidal harmonic function, which has the argument ηⱼ and ηₖ as shown here:

$${η}_{j} = \frac{{ρ}^{2} + {ρ}_{j}^{2} + {z}^{2}}{2 ρ {ρ}_{j}}$$

$${η}_{k} = \frac{{ρ}^{2} + {ρ}_{k}^{2} + {z}^{2}}{2 ρ {ρ}_{k}}$$

With reference to a coordinate system placed at the center of the concentric loops, such that loops all lie within the XY-plane, ρ measures radial distance from coil axis to a point within the specimen while z measures distance from the coil plane to the same point within the specimen.

The coil-loss model introduces electrical conductivity σ̆(\vec{r}) as a function of position. The integrals can be evaluated using a finite element mesh to generate the conductivity distribution for a plurality of coil property measurements as will be discussed in more detail below.

The coil-loss model relating a coil loss measurement at a particular coil position and orientation to an entire electrical conductivity distribution is actually a 3D convolution model. This can be seen from recasting the model into the a frame defined by a coil center—with the coil's Z-axis perpendicular to the coil plane, while the coil's X-axis and Y-axis lie within the coil plane. Letting the vector c locate the coil center relative to the lab frame origin, and the vector \vec{r_{c}} locate the field point in the coordinate system (CS) of the coil, a revised form for the model is as follows:

Z(\vec{c})=∫σ₁(\vec{c}+\tilde{R}\vec{r}_{c})G(\vec{r}_{c})dx_{c}dy_{c}dz_{c}=∫σ₁(\vec{r})G(\tilde{R}^{T}(\vec{r}−\vec{c}))dxdydz

In the above, the function G(\vec{r_{c}}) is recognized as the kernel of the convolution integral and can be defined as follows:

$$G ( {\vec{r}}_{c} ) = \frac{{μ}^{2} {ω}^{2}}{4 {π}^{2}} \frac{1}{ρ} {\sum }_{j , k} \sqrt{{ρ}_{j} {ρ}_{k}} {Q}_{1 / 2} ( {η}_{j} ) {Q}_{1 / 2} ( {η}_{k} )$$

The kernel function can be rapidly evaluated through the use of a hypergeometric series for toroidal functions.

As will be discussed in detail below, the coil-loss model and kernel function can be used in the inversion of coil property measurements according to example embodiments of the present disclosure. For instance, the convolution integral can be discretized over a finite mesh representation of the specimen.

### Example Coil Designs for Magnetic Induction Tomography Imaging

An example coil design that approximates the coil contemplated by the example quantitative coil-loss model will now be set forth. A coil according to example aspects of the present disclosure can include a plurality of concentric conductive loops arranged in two-planes on a multilayer printed circuit board. The plurality of concentric conductive loops can include a plurality of first concentric conductive loops located within a first plane and a plurality of second concentric conductive loops located in a second plane. The second plane can be spaced apart from the first plane by a plane separation distance. The plane separation distance can be selected such that the coil approximates the single plane coil contemplated in the example quantitative analytical coil-loss model for magnetic induction tomography imaging disclosed herein.

In addition, the plurality of conductive loops can be connected in series using a plurality of connection traces. The plurality of connection traces can be arranged so that the contribution to the fields generated by the connection traces can be reduced. In this manner, the coil according to example aspects of the present disclosure can exhibit behavior that approximates a plurality of circular loops arranged concentric to one another and located in the same plane.

FIG. 4 depicts an example coil 200 used for magnetic induction tomography imaging according to example aspects of the present disclosure. As shown, the coil 200 includes ten concentric conductive loops. More particularly, the coil 200 includes five first concentric conductive loops 210 disposed in a first plane and five second concentric conductive loops 220 disposed in a second plane. The first and second concentric conductive loops 210 and 220 can be 1 mm or 0.5 mm copper traces on a multilayer printed circuit board. In one example implementation, the radii of the five concentric conductive loops in either plane are set at about 4 mm, 8 mm, 12 mm, 16 mm, and 20 mm respectively. Other suitable dimensions and spacing can be used without deviating from the scope of the present disclosure.

As shown, each of the plurality of first concentric conductive loops 210 is disposed such that it overlaps one of the plurality of second concentric conductive loops 220. In addition, the first concentric conductive loops 210 and the second concentric conductive loops 220 can be separated by a plane separation distance. The plane separation distance can be selected such that the coil 200 approximates a single plane of concentric loops as contemplated by the quantitative analytical coil-loss model. For instance, the plane separation distance can be in the range of about 0.2 mm to about 0.7 mm, such as about 0.5 mm.

The plurality of first conductive loops 210 can include a first innermost conductive loop 214. The first innermost conductive loop 214 can be coupled to an RF energy source. The plurality of second conductive loops 220 can include a second innermost conductive loop 224. The second innermost conductive loop 224 can be coupled to a reference node (e.g. a ground node or common node).

The coil further includes a plurality of connection traces 230 that are used to connect the first concentric conductive loops 210 and the second concentric conductive loops 220 in series. More particularly, the connection traces 230 couple the plurality of first concentric conductive loops 210 in series with one another and can couple the plurality of second concentric conductive loops 220 in series with one another. The connection traces 230 can also include a connection trace 235 that couples the outermost first concentric conductive loop 212 with the outermost second concentric conductive loop 214 in series.

As shown in more detail in FIG. 5, the connection traces 230 can be arranged such that fields emanating from the connection traces oppose each other. More particularly, the connection traces 230 can be radially aligned such that a current flow of one of the plurality of connection traces located in the first plane is opposite to a current flow of one of the plurality of connection traces located in the second plane. For instance, referring to FIG. 5, connection trace 232 arranged in the first plane can be nearly radially aligned with connection trace 234 in the second plane. A current flowing in connection trace 232 can be opposite to the current flowing in connection trace 234 such that fields generated by the connection traces 232 and 234 oppose or cancel each other.

As further illustrated in FIG. 5, each of the plurality of first conductive loops 210 and the second conductive loops 220 can include a gap 240 to facilitate connection of the conductive loops using the connection traces 230. The gap can be in the range of about 0.2 mm to about 0.7 mm, such as about 0.5 mm.

The gaps 240 can be offset from one another to facilitate connection of the plurality of concentric conductive loops 210 and 220 in series. For instance, a gap associated with one of the plurality of first concentric conductive loops 210 can be offset from a gap associated with another of the plurality of first concentric conductive loops 210. Similarly, a gap associated with one of the plurality of second concentric conductive loops 220 can be offset from a gap associated with another of the plurality of second concentric conductive loops 220. A gap associated with one of the first concentric conductive loops 210 can also be offset from a gap associated with one of the plurality of second concentric conductive loops 220. Gaps that are offset may not be along the same axis associated with the coil 200.

The coil 200 of FIGS. 4 and 5 can provide a good approximation of the coil contemplated by the quantitative analytical coil-loss model for magnetic induction tomography imaging. In this way, coil property measurements using the coil 200 can be used to generate three-dimensional electromagnetic property maps of specimens of interest (e.g. human tissue specimens).

### Example Circuit for Obtaining Coil Property Measurements

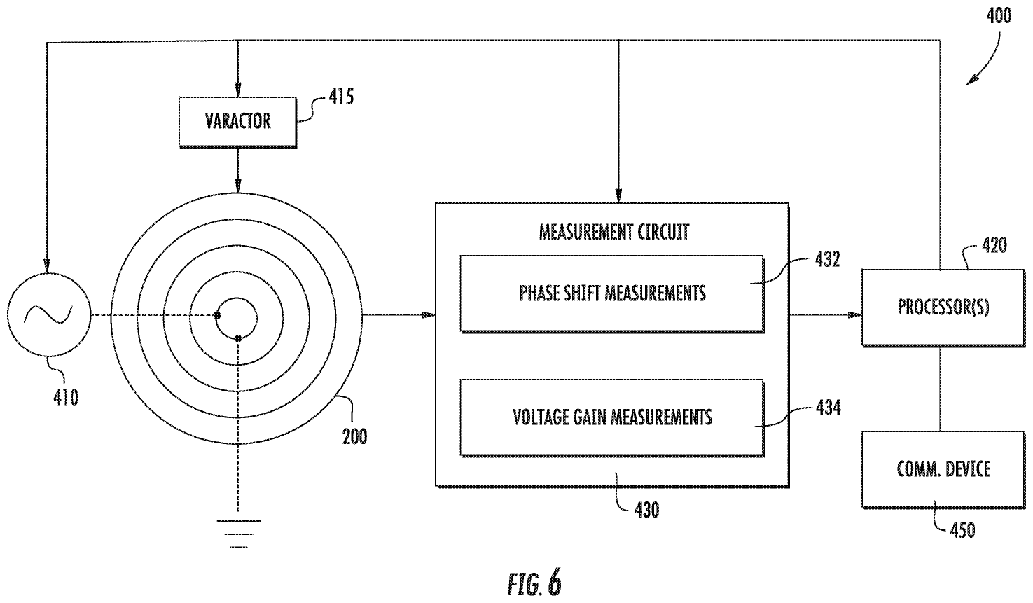

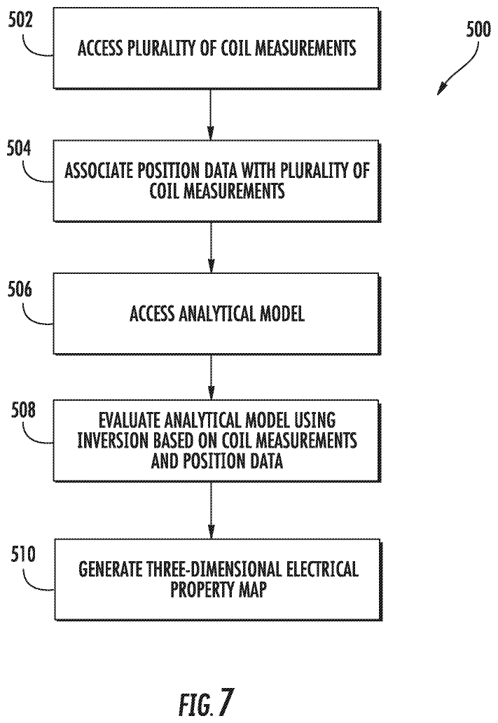

FIG. 6 depicts a diagram of an example circuit 400 that can be used to obtain coil property measurements using the coil 200 of FIGS. 4 and 5. As shown, the circuit 400 of FIG. 6 includes an RF energy source 410 (e.g. an oscillator circuit) configured to energize the coil 200 with RF energy. The RF energy source 410 can be a fixed frequency crystal oscillator configured to apply RF energy at a fixed frequency to the coil 200. The fixed frequency can be, for instance, about 12.5 MHz. In one example embodiment, the RF energy source 410 can be coupled to an innermost concentric conductive loop of the plurality of first concentric conductive loops of the coil 200. The innermost concentric conductive loop of the plurality of second concentric conductive loops of the coil 200 can be coupled to a reference node (e.g. common or ground).

The circuit 400 can include one or more processors 420 to control various aspects of the circuit 400 as well as to process information obtained by the circuit 400 (e.g. information obtained by measurement circuit 430). The one or more processors 420 can include any suitable processing device, such as digital signal processor, microprocessor, microcontroller, integrated circuit or other suitable processing device.

The one or more processors 420 can be configured to control various components of the circuit 400 in order to capture a coil loss measurement using the coil 200. For instance, the one or more processors 420 can control a varactor 415 coupled in parallel with the coil 200 so as to drive the coil 200 to resonance or near resonance when the coil 200 is positioned adjacent a specimen for a coil property measurement. The one or more processors 420 can also control the measurement circuit 430 to obtain a coil property measurement when the coil 200 is positioned adjacent the specimen.

The measurement circuit 430 can be configured to obtain coil property measurements with the coil 200. The coil property measurements can be indicative of coil losses of the coil 200 resulting from eddy currents induced in the specimen. In one implementation, the measurement circuit 430 can be configured to measure the real part of admittance changes of the coil 200. The real part of admittance changes of the coil 200 can be converted to real part of impedance changes of the coil 200 as the inverse of admittance for purposes of the analytical coil-loss model.

The admittance of the coil 200 can be measured in a variety of ways. In one embodiment, the measurement circuit 430 measures the admittance using a phase shift measurement circuit 432 and a voltage gain measurement circuit 434. For instance, the measurement circuit 430 can include an AD8302 phase and gain detector from Analog Devices. The phase shift measurement circuit 432 can measure the phase shift between current and voltage associated with the coil 200. The voltage gain measurement circuit 434 can measure the ratio of the voltage across the coil 200 with a voltage of a sense resistor coupled in series with the coil 200. The admittance of the coil 200 can be derived (e.g., by the one or more processors 420) based on the phase and gain of the coil 200 as obtained by the measurement circuit 430.

Once the coil property measurements have been obtained, the one or more processors 420 can store the coil property measurements, for instance, in a memory device. The one or more processors 420 can also communicate the coil property measurements to one or more remote devices for processing to generate a three-dimensional electromagnetic property map of the specimen using communication device 440. Communication device 440 can include any suitable interface or device for communicating information to a remote device over wired or wireless connections and/or networks.

### Example Methods for Magnetic Induction Tomography Imaging

FIG. 7 depicts a process flow diagram of an example method (500) for magnetic induction tomography imaging according to example aspects of the present disclosure. The method (500) can be implemented by one or more computing devices, such as one or more computing devices of the map generation system 140 depicted in FIG. 1. In addition, FIG. 7 depicts steps performed in a particular order for purposes of illustration and discussion. Those of ordinary skill in the art, using the disclosures provided herein, will understand that the steps of any of the methods disclosed herein can be modified, omitted, rearranged, performed simultaneously, adapted, or expanded in various ways without deviating from the scope of the present disclosure.

At (502), the method can include accessing a plurality of coil property measurements obtained using a MIT device at a plurality of different discrete locations relative to the specimen. For instance, the plurality of coil property measurements can be accessed from a memory device or can be received from a coil device having a single coil configured for obtaining the coil property measurements. The coil property measurements can be coil loss measurements captured by a single coil when the single coil is energized with RF energy and placed adjacent a specimen at one of the plurality of discrete locations.

In one implementation, the single coil can include a plurality of concentric conductive loops. For instance, the single coil can have a plurality of first concentric conductive loops arranged in a first plane and a plurality of second concentric conductive loops arranged in a second plane. The plurality of concentric conductive loops can be connected using connection traces arranged so as to have a reduced impact on the field created by the coil.

The coil property measurements can be obtained at a plurality of discrete positions relative to the specimen. Each coil property measurement can be taken at a different discrete position relative to the specimen. A greater number of coil property measurements can lead to increased accuracy in generating a three-dimensional electromagnetic property map from the coil property measurements.

In a particular embodiment, the coil property measurements can include a plurality of different data sets of coil property measurements. Each of the data sets can be built by conducting a plurality of coil property measurements using a single coil. The single coil can be different for each data set. For instance, each data set can be associated with a single coil having a different overall size and/or outer diameter, relative to any of the other single coils associated with the other data sets. The data sets can be obtained at different times. The data sets can be collectively processed according to example aspects of the present disclosure to generate a three-dimensional map of an electrical property distribution of the specimen as discussed below.

At (504) of FIG. 7, the method includes associating position data with each of the plurality of coil property measurements. The position data for each coil property measurement can be indicative of the position and/or orientation of the single coil relative to the specimen when the coil property measurement was performed. The position data can be associated with each coil property measurement, for instance, in a memory device of a computing system.

The position data can be obtained in a variety of ways. In one implementation, the position data can be obtained for each measurement from data associated with a positioning system configured to determine a position and/or orientation of the MIT device as the MIT device is used to obtain measurements. In addition, signals from one or more sensors (e.g. one or more motion sensors and one or more depth sensors) associated with the MIT device can be also used to determine the position data for a coil property measurement.

At (506), the method includes accessing an analytical coil-loss model defining a relationship between coil property measurements obtained by the single coil and an electromagnetic property of the specimen. For instance, the analytical coil-loss model can be accessed, for instance, from a memory device. In one particular implementation, the analytical coil-loss model correlates a change in an impedance of a single coil having a plurality of concentric conductive loops with a conductivity distribution of the specimen. More particularly, the analytical coil-loss model can correlate the change in impedance of a single coil with a variety of parameters. The parameters can include the conductivity distribution of the specimen, the position and orientation associated with each coil loss measurement, and the geometry of the coil (e.g., the radius of each of the concentric conductive loops). Details concerning an example quantitative coil-loss model were provided in the discussion of the example quantitative analytical coil-loss model for a single coil discussed above.

At (508), the method includes evaluating the analytical coil-loss model based on the plurality of coil property measurements and associated position data to determine an electromagnetic property distribution. More particularly, an inversion can be performed using the coil-loss model to determine a conductivity distribution that most closely leads to the plurality of obtained coil property measurements. In one example aspect, the inversion can be performed by discretizing the specimen into a finite element mesh. The shape and resolution of the finite element mesh can be tailored to the specimen being analyzed.

More particularly, a plurality of candidate electromagnetic property distributions can be evaluated using a penalized objective function having two terms: (1) an error term and (2) a penalty term. As discussed in more detail below, in some embodiments, a penalty term for each node in the finite element mesh can be independently weighted. For instance, a regularization matrix can be used to independently weight penalty terms for individual nodes in the finite element mesh. The objective function can be converted to standard form. The standard form objective function can be evaluated using SVD to generate an SVD objective function which is more amenable for algebraic solution. The SVD objective function can be expanded using Lagrange mulitpliers to provide a Lagrange objective function. Non-negative constraints can be enforced using Lagrange multipliers. The Lagrange objective function can be solved using an active set and a plurality of iterations. During each iteration, a global regularization parameter associated with the penalty term can be stepped through a sequence of singular values until a solution error is within a threshold of a measurement error. The sequence of singular values can progress from largest to smallest. Details concerning the example inversion and independent selection of weights for the penalty term (e.g., regularization matrix) are provided below.

At (510), a three-dimensional electromagnetic property map can be generated based on the electromagnetic property distribution identified using the inversion algorithm. The three-dimensional property map can provide an electromagnetic property distribution (e.g., a conductivity distribution) for a plurality of three-dimensional points associated with the specimen. Two-dimensional views along cross-sections of the three-dimensional electromagnetic property map can then be captured and presented, for instance, on a display device. Three-dimensional views of the electromagnetic property map can also be generated, rotated, and presented, for instance, on a display device.

### Example Inversion of Measurements for Determining Distribution

The discretized convolution integral of the coil loss model can lead to a prediction of coil loss measurements from a conductivity vector \vec{x} consisting of n conductivity values on a finite element (FE) mesh. For the application to single-coil MIT, coil loss measurements are obtained at m scan positions in the vicinity of a conductive target. Coil loss prediction is given by the vector Ã\vec{x}, where the matrix Ã is m×n and includes components that are determined according to coil position and orientation. Measurements can be few in number, such that m<n, giving an underdetermined system.

Because conductivity is non-negative, we require that xᵢ≥0 for each i. Since scanning inevitably produces many similar measurements, the matrix Ã can be ill-conditioned (large condition number). As a result, conductivity can be found by minimizing or reducing to a near optimum value a penalized objection function (e.g., a penalized 2-norm objective function) subject to non-negativity:

min ½∥Ã\vec{x}−\vec{b}∥₂²+½τ²∥\tilde{D}(\vec{x}−\vec{β})∥₂²s.t. \vec{x}≥0 (1)

The penalized objective function can have two terms: (1) an error term (first part); and (2) a penalty term (second part).

The solution to the problem of minimizing or reducing to a near optimum value the penalized objective function (1) can be made somewhat more flexible, without requiring additional computational effort, by permitting each node to be penalized independently of the other nodes. As discussed in detail below, this can be accomplished via the invertible, diagonal regularization matrix \tilde{D}, with diagonal entries dⱼ>0. In order for the weights in the diagonal matrix to produce some benefit to the solution process, guidance on selection of the components of \tilde{D} can be implemented as discussed in detail below. For instance, the coil-loss model might inherently weight some members of a solution set more so than others. Regularization matrix \tilde{D} offers the ability to compensate. Furthermore, if some solution members tend to contribute very little to a predicted bᵢ, or should have little impact on the error term of the penalized objective function, then these could be penalized heavily to ensure a given xᵢ remains well inside an expected interval through appropriate assignment of βⱼ and dⱼ.

Commonly \vec{β}=0 but here advantage is taken of the fact that the solution can lie within some interval 0≤xᵢ≤5 S/m rather than an unbounded domain. For example, electrical conductivity within human tissues can be less than ˜5 S/m, suggesting that \vec{β} ought to be set somewhere inside this expected interval-leading to a “shifted penalty.” Thus, the regularization matrix of the penalty term not only can promote a well-conditioned problem with a unique solution, but can lead to a greater penalty when xᵢ≠\vec{β}. If the regularization parameter τ is made large compared to the largest singular values of Ã, then solutions tend to be heavily

localized around \vec{β}. Thus, \vec{β} can be set >0 so that the number of negative xᵢ values is reduced in the absence of constraints, while τ is set to a non-zero value within the range of the singular values associated with Ã. Non-negativity can be addressed using an active set method, but can be made more efficient as a result of the shifted penalty, which tends to reduce the number of needed constraints, and thus the size of the active set.

Under these definitions, the objective function (1) can be transformed to standard form:

\vec{z}=\tilde{D}\vec{x}; \vec{ζ}=\tilde{D}\vec{β} (2)

The penalized objective function (1) becomes a standard form objective function:

min ½∥Ã\tilde{D}⁻¹\vec{z}−\vec{b}∥₂²+½τ²∥\vec{z}−\vec{ζ}∥₂²s.t. \vec{z}≥0 (3)

As shown, the objective function (3) is in standard form with the exception of the shifted penalty. Objective function (3) sows how matrix Ã is preprocessed by the inverse of regularization matrix \tilde{D}⁻¹, which can balance the role of each component of the solution. Converting to standard form can be accomplished in other ways without deviating from the scope of the present disclosure. A benefit of converting to standard form is improved efficiency in obtaining a solution.

SVD can be applied to the matrix product Ã\tilde{D}⁻¹ in objective function (3) above as follows:

Ã\tilde{D}⁻¹=Ũ\tilde{S}\tilde{V}^{T} (4)

Orthogonal matrices and singular values can be modified due to the pre-multiplication of Ã by the inverse of \tilde{D} so that solution components have a more equitable chance to contribute to the measurement vector. Additional assignments can be made as follows:

\vec{x}=\tilde{V}\vec{y}; \vec{b}=Ũ\vec{b}′; ζ\tilde{V}\vec{β}′ (5)

This yields a minimization problem for a modified objective function that is especially suitable for inversion since it is amenable to an algebraic solution:

min ½∥\tilde{S}\vec{y}−\vec{b}′∥₂²+½τ²∥\vec{y}−\vec{β}′∥₂² s.t. \vec{x} or \vec{z}≥0 (6)

SVD objective function (6) can be expanded and non-negative constraints can be set for an active set (e.g. some select members of the solution set) {x_{i{k}}; k=1, 2, . . . , K} to achieve the Lagrange objective function:

$$L ( \vec{y} , \vec{λ} ) = \frac{1}{2} {\sum }_{i = 1}^{r} {( {s}_{i} {y}_{i} - {b}_{i}^{′} )}^{2} + \frac{1}{2} {\sum }_{i = r + 1}^{m} {( {b}_{i}^{′} )}^{2} + \frac{1}{2} {τ}^{2} {\sum }_{i = 1}^{n} {( {y}_{i} - {B}_{i}^{′} )}^{2} + {\sum }_{k = 1}^{K} {λ}_{k} {x}_{l ( k )} \qquad ( 7 )$$

Assuming that r<m<n, wherein the matrix Ã has rank r, when r=m (usually), the second term on the right side of the Lagrange objective function (7) is absent. Constraints, enforced via Lagrange multipliers {λₖ}) are only applied to those points belonging to the active set. To establish an initial active set, the Lagrange objective function (7) is first minimized without constraints while \vec{β} is chosen to deliberately reduce the size of the active set. Nodes with negative values are identified and placed in the active set for the first iteration following initialization. At each new iteration, Lagrange multipliers are examined so that a constraint is released when positive but maintained if negative. Nodes that are not in the active set continue to be examined and placed in the active set if there values are found to be negative.

Equations can be written for each of the unknowns in the Lagrange objective function (7) by setting a partial derivative of the objective function with respect to each of yⱼ and λᵢ to zero to yield the following:

$$\begin{matrix}{y}_{j} = \{ \begin{matrix}\frac{{s}_{j} {b}_{{ }^{j}}^{′} + {r}^{2} {β}_{j}^{′} - {\sum }_{k = 1}^{K} {λ}_{k} {V}_{l ( k ) j} / {d}_{l ( k )}}{{s}_{j}^{2} + {r}^{2}} ; j \leq r \\ \frac{{τ}^{2} {β}_{j}^{′} - {\sum }_{k = 1}^{K} {λ}_{k} {V}_{t ( k ) j} / {d}_{k}}{{τ}^{2}} ; r < j \leq n\end{matrix} ; k \to l mapping & ( 8 ) \\ {x}_{l ( q )} = {\sum }_{j = 1}^{n} \frac{{V}_{l ( q ) j}}{{d}_{l ( q )}} {y}_{j} = 0 ; q \to l mapping implied & ( 9 )\end{matrix}$$

Substituting (8) into (9) obtains Lagrange multipliers from a relatively small set of linear equations:

$${\sum }_{k = 1}^{K} {P}_{qk} {λ}_{k} = {\sum }_{j = 1}^{n} \frac{{V}_{l ( q ) j}}{{d}_{l ( q )}} {y}_{j}^{0} ; \\ q = 1 , 2 , … , K ( \text{∼} 1500 ) << n ( \text{∼} 11 , 000 ) \qquad ( 10 )$$

The elements for the symmetric matrix, P_{qk}, are given by:

$${P}_{qk} = \frac{1}{{d}_{l ( q )} {d}_{l ( k )}} {\sum }_{j = 1}^{n} \frac{{V}_{l ( q ) j} {V}_{l ( k ) j}}{{s}_{j}^{2} + {τ}^{2}} ; \\ {s}_{j} = 0 if j > r ; q , k = 1 , 2 , 3 , … , K \qquad ( 11 )$$

The vector components of \vec{y}⁰ are just the unconstrained solution, obtained by setting all Lagrange multipliers to zero in equation (8). Equation (10) can be solved using an LDL^{T} decomposition method, and can be kept smaller in size if \vec{β} is chosen within the expected solution interval and the regularization parameter τ is kept nearer to the larger singular values in the set {sⱼ}. By choosing τ nearer the middle of the set of singular values, the current solution approach can behave similarly to a truncated SVD method since those singular values which are much
smaller than τ have little or no effect on the solution (filtering)—note that all singular values can be used here and regularization can be controlled by choice of τ rather than truncation. Since the active set may change “membership” with each iteration, matrix \tilde{P} may be recomputed, but only for those components new to the active set.

Subsequent to solving (1), the Lagrange multipliers are returned to (8) to get a new solution (either \vec{z} or \vec{x}). At that point, both the new solution and Lagrange multipliers can be examined to establish a new active set for the next iteration. Convergence can occur in a finite number of steps. For MIT applications, convergence can occur after about 5 to about 7 iterations. Convergence can be determined by the attainment of a stable active set.

This approach can require a choice for \vec{β}, which can establish a location where the penalty term of the objective function is zero. Optionally, individual components of the penalty term may be forced to zero at customized locations, without requiring that the full penalty term is zero at a common location.

In one example implementation, a uniform solution is chosen that sets all solution components to an identical value β₀ (where the entire penalty term is zero). This can be found by minimizing the residual with respect to β₀:

$${β}_{0} = \frac{{\sum }_{i = 1}^{m} {b}_{i} {\sum }_{j = 1}^{n} {A}_{ij}}{{\sum }_{i = 1}^{m} {( {\sum }_{j = 1}^{n} {A}_{ij} )}^{2}} \qquad ( 12 )$$

Other approaches can be used without deviating from the scope of the present disclosure. For instance, \vec{β} can be modified as the iteration process continues with the idea that the next solution is more penalized as it departs further from a prior solution.

According to example aspects of the present disclosure, a value for the global regularization parameter τ can be set using a discrepancy principle. This can require some reasonable estimate is available for the error associated with measurement vector \vec{b}:

∥\vec{b}−\vec{b}ₜᵣᵤₑ∥≅ε (13)

The error ε can be available from experimental error. The regularization parameter τ can be decreased along a sequence tracking singular values from the SVD of equation (4), perhaps jumping several singular values at a time, until the following condition is approximately met:

∥Ã\vec{x}−\vec{b}∥≥ε (14)

This is an acceptable stopping condition for the model as it is not necessary for prediction with greater precision than what is available via experiment.

### Example Independent Setting of Weights for Regularization Matrix

As discussed, the diagonal matrix \tilde{D} provides an opportunity to selectively penalize contributions from individual solution components. Essentially, the kernel function in the convolution integral naturally weights regions of space according to their contribution to the measured coil loss. Thus, plots of the kernel function can be helpful in guiding how the components of \tilde{D} are chosen.

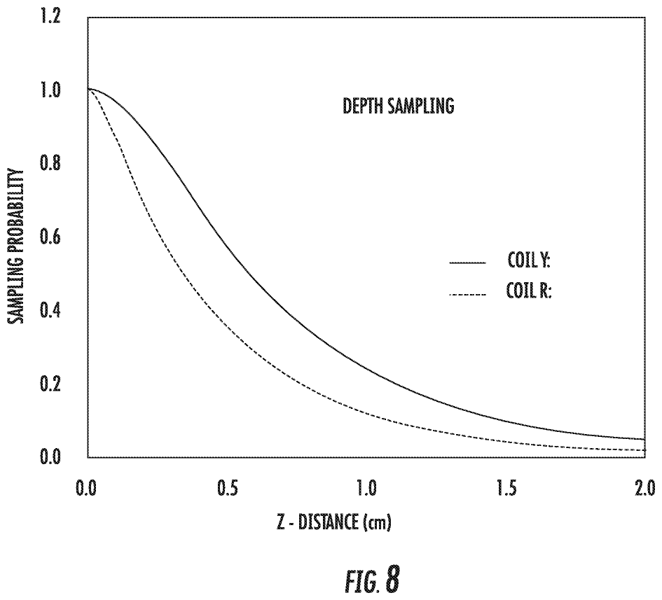

FIG. 8 provides kernel function plots for two different coil designs. Coil ‘R’ includes five circular concentric loops in each of two layers (ten total)—spacing is 0.5 mm, so that all loops are considered to effectively lie in the same plane. The loops have radii of 4, 8, 12, 16 and 20 mm. Coil ‘R’ inductance was shown previously to be 2.155 pH. A second coil ‘Y’ is provided for comparison purposes. Coil ‘Y’ includes 4 layers of loops, all set at a radius of 20 mm, with a spacing of ˜0.3 mm between layers. Spacing is considered to be sufficiently small that they are considered to be in the same plane. Coil ‘Y’ inductance is computed to be ˜1.8 pH.

FIG. 8 plots the kernel function as a function of distance from the coil along the Z-axis for coil ‘R’ and coil ‘Y”. Plots are obtained at a radial distance of 15 mm from the coil axis. FIG. 8 shows how spatial weighting varies with distance from the coil plane. Regardless of coil type, the decay with Z-distance in FIG. 8 is nearly exponential, except in the immediate vicinity of the coil plane. FIG. 8 indicates that greater weight or importance is given to those points nearest to the coil plane, giving them an advantage in a noisy environment, so that the solution from any inversion scheme will tend to favor points closest to the coil plane if nothing is done to relieve the bias.

According to example aspects of the present disclosure a simple procedure to help mitigate the bias applies an exponential decrease of the regularization weights in \tilde{D} as solution components become associated with points farther from the coil plane or target boundary (e.g., a boundary associated with a specimen). In some embodiments the regularization weights \tilde{D} are applied as follows:

dⱼ=exp(−ηxⱼ/zₘₐₓ) (19)

Where finite element mesh thickness is given as zₘₐₓ, zⱼ is the distance from the target boundary to the node location, and η is a decay parameter meant to control the extent of bias mitigation.

In some embodiments, plots of the kernel function (e.g., as shown in FIG. 8) can be used to determine the weights of the penalty term for each node.

### Simulation Results

Simulations were performed with respect to two virtual phantoms. The phantoms include buried thick strips of altered conductivity. The first phantom 602 has low conductivity strips (˜0.0 S/m) immersed in a higher conductivity matrix (˜1.0 S/m). The second phantom has higher conductivity strips immersed in a lower conductivity matrix.

In the first phantom 602, two thick strips of very low conductivity material, embedded in background material of 1.0 S/m, are located within a 16.5 cm×16.5 cm×3.0 cm slab phantom as shown in FIG. 9. These low-conductivity strips are defined by regions having conductivity beneath 0.1 S/m. The two strips each run parallel to the Y-axis, located at X=4.5 cm and X=12.0 cm; and with Z=1.0 cm in either case. Using the definition that strip conductivity is <0.1 S/m, then strip thickness is ˜1.8 cm, while strip width for each is ˜3.8 cm. Separation between strips is ˜4.5 cm. Outside of a small region where conductivity is near 0.0 S/m, a squared Gaussian function blends the low conductivity strip with the bulk region at 1.0 S/m.

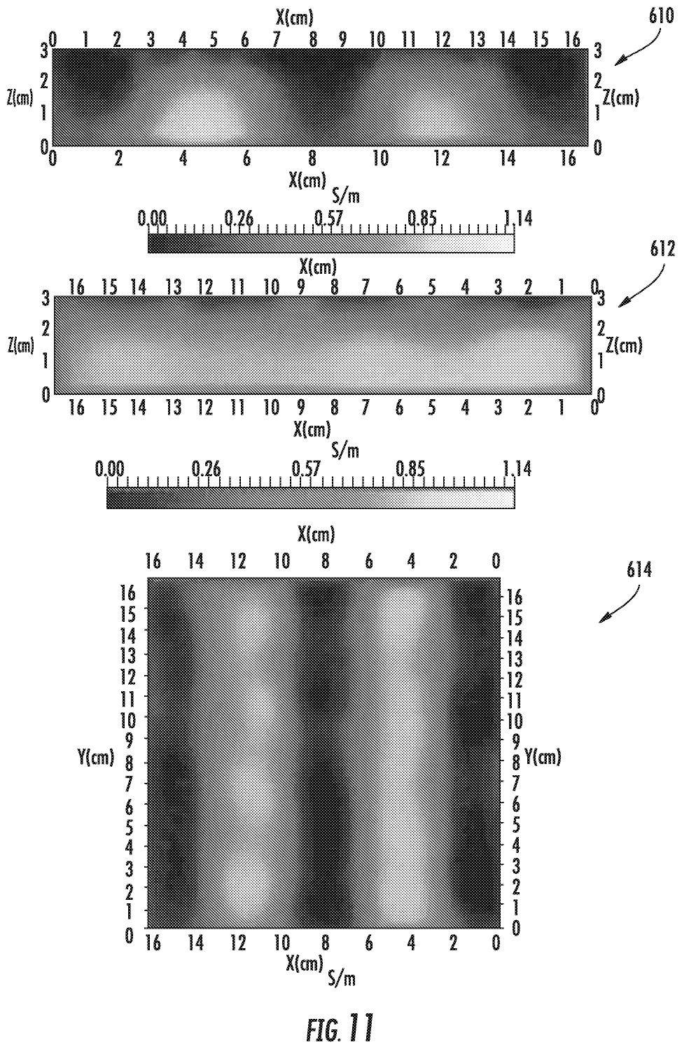

The second phantom 604 is just the inverse of the first phantom and includes buried higher conductivity material (1.0 S/m) embedded in low conductivity matrix. The second phantom is depicted in FIG. 10.

Simulated coil loss measurement data was obtained for the phantoms 602, and 604 as follows. The analytical formula given by the coil loss model discussed above is used—giving loss in a coil of type ‘R’. The sample-containing space of the phantom is subdivided into 10 layers of equal thickness. For numerical integration, the total number of elements is set to 41,180 and integration on any element follows a 9 point quadrature rule. Integrations are done for each of 486 coil locations above the slab, on a 9×9×6 lattice. Coil loss is computed for 81 locations on each of the six horizons on a regular grid. Odd layers, starting with the first, all share the same grid, while even numbered horizons share a common grid that is shifted by half a grid interval from the odd numbered horizons.

Sampling horizons are equally spaced from each other over a span of 6.0 mm above the slab. The first sampling horizon keeps the coil parallel to the slab and spaced 2.0 mm above the slab. All other sampling horizons allowed for a randomly tilted coil, with axis tilted as much as 5 degrees from vertical, which alters the height of coil center. Allowing for coil tilt when the coil is clear of the slab is done simply to better mimic an actual hand scan where coil tilt is difficult to avoid. Gaussian noise is added to the simulated coil loss data to simulate noise. An error bound for the simulation was determined to be 0.0063 ohms.

The coil loss model was discretized into a finite element mesh having 20,080 elements over eight layers of equal thickness. A value for the global regularization parameter τ was chosen as one of the singular values, which decreases monotonically from a maximum value atj=1 to a smallest value at j=486 (number of measurements). For the phantom consisting of two buried strips of 1.0 S/m material, j=220 was selected, which gave an error norm of 0.0047 ohms, close to the target of 0.0063 ohms. The nodes are independently weighted as a function of distance from a target boundary as set forth in equation (19). The non-dimensional decay parameter used to independently weight the nodes in the penalty term (e.g., η in equation (19)) was set to 1.0, as suggested by exponential fits to the ‘R’ curve in FIG. 8. Convergence is obtained after 6 iterations of the active set method.

Three image slices are shown in FIG. 11, a transverse slice 610 midway along and normal to the Y-axis, a longitudinal slice 612 normal to the X-axis, and a Z-normal slice 614 midway along the Z-axis. To facilitate comparison, these same slice locations are used in FIGS. 11 and 1313.

Each slice in FIG. 11 indicates that image reconstruction from 486 measurements does a reasonably good job of faithfully representing the original phantom 602 in FIG. 9. Conductivity spans from 0.0 to about 1.0 S/m, as it should; conductivity stays near 0.0 S/m in the space separating the two strips, from the top of the phantom all the way to the bottom; and, features are arranged in spatially correct position. Of particular importance, variation of structural features with respect to depth track rather well those of the actual phantom, suggesting that the diagonal regularization matrix is performing as intended.

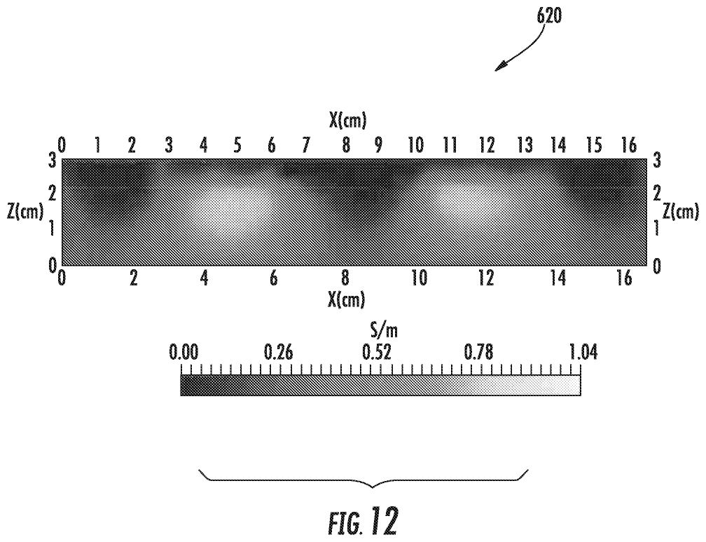

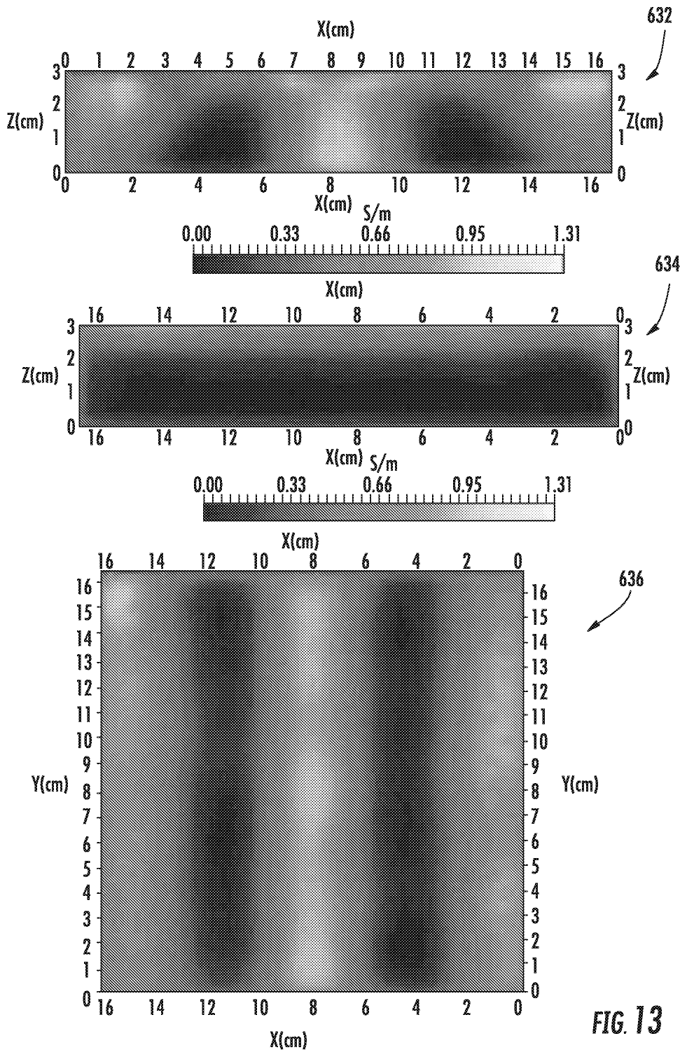

FIG. 12 depicts a longitudinal slice 620 resulting from setting the decay parameter for independently weighting the penalty term for nodes in the regularization matrix to zero (equal weighting). The effect is shown in the single slice 620, normal to the Y-axis (transverse to strips) in FIG. 12. Comparing the transverse slices 612 and 620 of FIGS. 11 and 12, it becomes clear that when solution components are equally weighted in the penalty term, computed conductivity in between or to either side of the strips struggles more so to stay near zero over the complete depth of the field compared with the case when decay parameter is set to 1.0. The effect is perhaps more noticeable inside the strips, where in FIG. 12, conductivity is considerably higher near the upper portions of the strips. If the decay parameter is increased to 1.5, conductivity inside the strips is skewed more toward the bottom, suggesting that 1.0 was near optimal.

The second phantom 604 is perhaps more challenging for image reconstruction, since now, the algorithm has to “see through” the higher conductivity matrix just to reveal the low conductivity strips. Because there is so much matrix material at 1.0 S/m, at least compared to the strips of the first phantom, the variation in signal measurements (coil loss) as the coil completes its scan is less, and added noise would tend to have the effect of corrupting the natural variation in coil loss. For this second phantom, our stopping condition led to τ value equal to the 210th singular value, similar to before, and a similar error norm value as well.

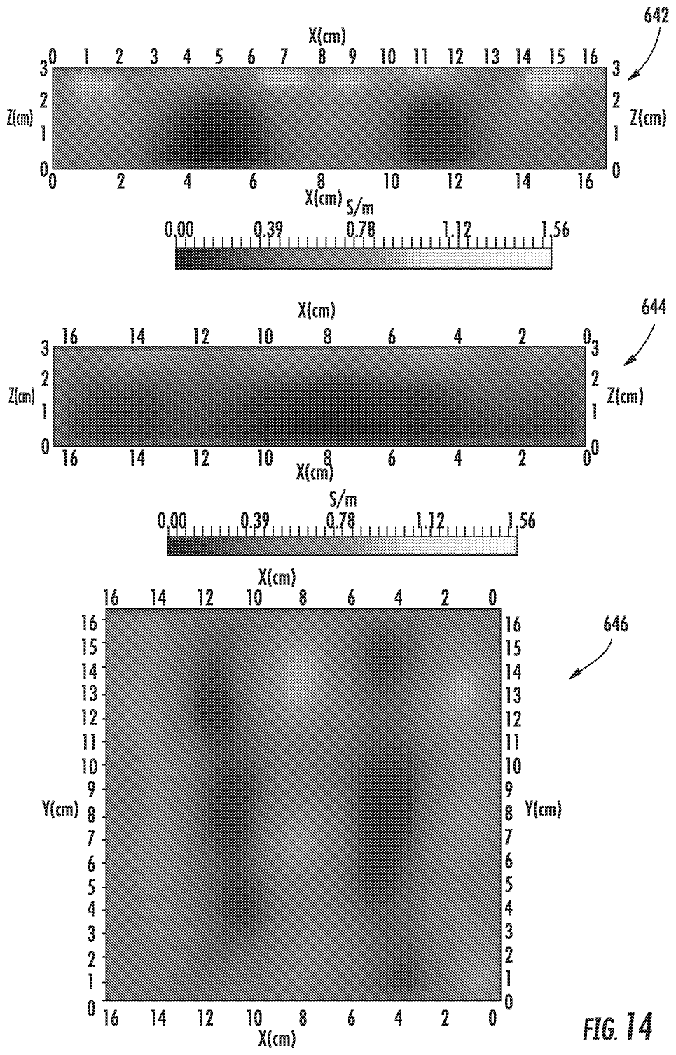

FIG. 13 depicts image slices 632, 634, and 636 that correspond to those of FIG. 11, but with an expected image inversion of the electrical conductivity distribution. Remarkably, the longitudinal slice along the length of the low-conductivity strip cleanly reveals the thin layer of high conductivity material just above the strip's entire length.

The reconstructed images shown in FIG. 13 for buried low conductivity strips again do a reasonable job of faithfully matching the features of the phantom 604 shown in FIG. 10. Though not apparent in the slices of FIG. 13, very few locations inside the full 3D image exhibit conductivity near the maximum of 1.31 S/m shown on the side scale-just a spot near a corner of the upper boundary where sampling is least adequate. Thus, most regions outside the strips are near the 1.0 S/m maximum conductivity, as expected. Perhaps most satisfying is the ability to depth-resolve the slab, especially the electrical conductivity distribution inside the strips and in the space between them.

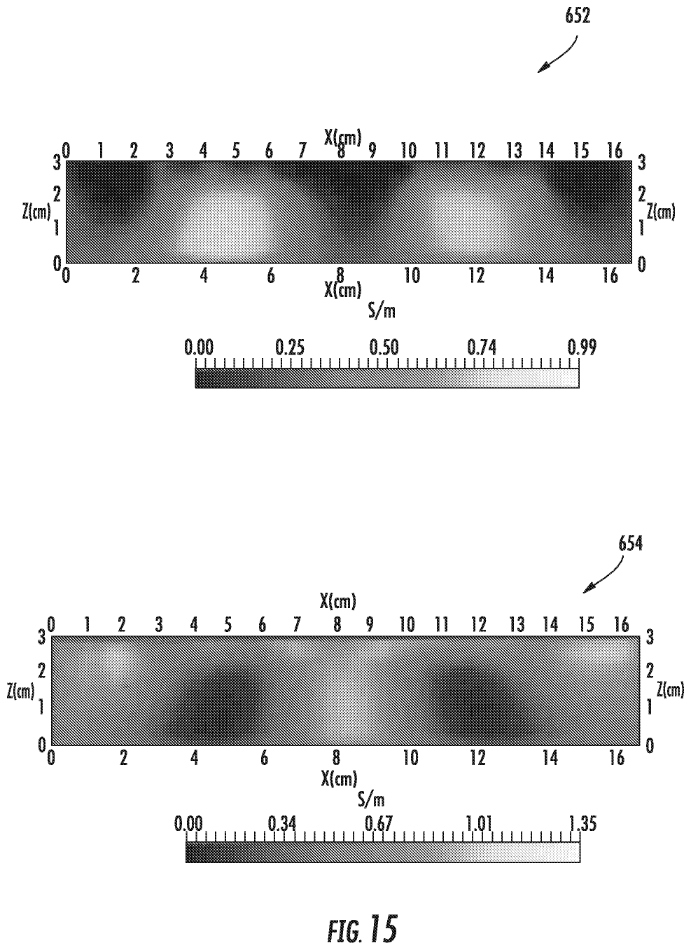

To understand how noise impacts the image reconstruction process, noise was increased 5× for the phantom comprised of low conductivity strips buried in 1.0 S/m background, to an admittance precision of ±0.05 S. This is double the noise level of newer instrumentation in current testing, and corresponds to a coil loss uncertainty of ±0.00143 ohms, or a target error norm of 0.0315 ohms (for 486 measurements). Image slices 642, 644, and 646 are shown in FIG. 14, and may be directly compared to those in FIG. 13.

As expected, the inversion is not immune to noise. Note that geometrical distortions start to develop, which are perhaps most noticeable in the Z-normal slice (image c) where strips become wavy. Nevertheless, features continue to be correctly located and readily identifiable relative to the phantom 604 shown in FIG. 10 10.

A weighting scheme for independently weighting the penalty term different than the exponential weights proposed in equation (19) is considered. Weights are based on the kernel function discussed above according to the following:

dⱼ=G(ρ,z_{scale}); z_{scale}=ηzⱼ/zₘₐₓ (21)

The scaling parameters have the same significance as before with just a different function than exponential. An important difference is that a distance from the coil axis is chosen for creating weights. Here, the chosen distance is simply the location where G would be maximum along the radial direction. Reconstruction based upon equation (21) sets the decay parameter η=0.3 and with stopping conditions as previously described, singular values having index equal to 220 and 210 were used for buried 1.0 S/m and 0.0 S/m strips respectively.

Image slices 652 and 654 for the above simulation are shown in FIG. 15. The images shown in FIG. 15 do not appear much different than the corresponding images given in FIGS. 11 and 13. There is perhaps a greater tendency for the “kernel-weighted” images to better preserve the square cross sections of the strips.

While the present subject matter has been described in detail with respect to specific example embodiments thereof, it will be appreciated that those skilled in the art, upon attaining an understanding of the foregoing may readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art.

1. A method for imaging a tissue specimen using magnetic induction tomography, the method comprising:
accessing, by one or more processors, a plurality of coil property measurements obtained for a specimen using a single coil, each of the plurality of coil property measurements obtained with the single coil at one of a plurality of discrete locations relative to a specimen;
associating, by the one or more processors, coil position data with each of the plurality of coil property measurements, the coil position data indicative of the position and orientation of the single coil relative to the specimen for each coil property measurement;
accessing, by the one or more processors, a coil-loss model defining a relationship between coil property measurements obtained by the single coil and an electromagnetic property of the specimen; and
determining, by the one or more processors, an electromagnetic property distribution for the specimen by performing an inversion based at least in part on the coil-loss model and the coil property measurements,
generating, by the one or more processors, an electromagnetic property map of the specimen using the coil-loss model based at least in part on the electromagnetic property distribution.

2. The method of claim 1, wherein the inversion is based at least in part on a penalized objective function, the penalized objective function having a penalty term, wherein the penalty terms for individual nodes in a finite element mesh are independently weighted.

3. The method of claim 2, wherein the penalty terms for individual nodes are independently weighted based on a distance along a perpendicular axis relative to the coil from a target boundary or to account for bias in the coil-loss model.

4. The method of claim 2, wherein the inversion uses a regularization matrix to weight the penalty term for individual nodes based on a kernel function associated with the coil-loss model.

5. The method of claim 4, wherein the regularization matrix is a diagonal regularization matrix.

6. The method of claim 2, wherein the penalty term is quadratic about a uniform electromagnetic property solution.

7. The method of claim 4, wherein the inversion comprises converting the penalized objective function to standard form to generate a standard form objective function using an inverse of the regularization matrix.

8. The method of claim 7, wherein the inversion applies singular value decomposition (SVD) to the standard form objective function to generate an SVD objective function.

9. The method of claim 8, wherein the inversion expands the SVD objective function using Lagrange multipliers to achieve a Lagrange objective function

10. The method of claim 9, wherein the inversion enforces a non-negative constraint using one or more Lagrange multipliers.

11. The method of claim 9, wherein the inversion determines a solution for the Lagrange objective function using an active set and a plurality of iterations.

12. The method of claim 11, wherein the inversion steps a global regularization parameter associated with the penalty term through a sequence of singular values until a solution error is within a threshold of a measurement error.

13. The method of claim 12, wherein the sequence of singular values progresses from largest to smallest.

14. The method of claim 1, wherein each of the plurality of coil property measurements comprises a coil loss measurement indicative of a change in impedance of the single coil resulting from eddy currents induced in the specimen when the single coil is placed adjacent to the specimen and energized with radiofrequency energy.

15. The method of claim 1, wherein the electromagnetic property is conductivity.

16. The method of claim 1, wherein the method comprises outputting the electromagnetic property map as an image on a display device.

17. A system for generating an image of a property of a tissue specimen, the system comprising one or more processors and one or more memory devices, the one or more memory devices storing computer-readable instructions that when executed by the one or more processors cause the one or more processors to perform operations, the operations comprising:
accessing a plurality of signal measurements;
associating position data with each of the plurality of signal measurements;
accessing a convolution model defining a relationship between signal measurements and a property of the specimen; and
determining a property distribution for the specimen by performing an inversion based at least in part on the convolution model and signal measurements, wherein the inversion is based at least in part on a penalized objective function, the penalized objective function having a penalty term, wherein the penalty term for individual solution components are independently weighted;
generating a property map of the specimen based at least in part on the property distribution; and
outputting the property map as an image on a display device.

18. The system of claim 17, wherein the penalty terms for individual nodes are independently weighted based on a distance along a perpendicular axis relative to a measurement apparatus from a target boundary or are independently weighted to account for bias in the convolution model.

19. The system of claim 17, wherein the inversion uses a regularization matrix to weight the penalty term for individual nodes based on a kernel function associated with the convolution model, the regularization matrix being a diagonal regularization matrix, the inversion comprising converting the penalized objective function to standard form to generate a standard form objective function using an inverse of the regularization matrix.

20. The system of claim 19, wherein the inversion applies singular value decomposition (SVD) to the standard form objective function to generate an SVD objective function, the inversion expanding the SVD objection function using Lagrange multipliers to achieve a Lagrange objective function, the inversion enforcing a non-negative constraint using one or more Lagrange multipliers, the inversion determining a solution for the Lagrange objective function using an active set and a plurality of iterations, the inversion stepping a global regularization parameter associated with the penalty term through a sequence of singular values until a solution error is within a threshold of a measurement error, the sequence of singular values progressing from largest to smallest.
